(19) United States
(12) Patent Application Publication
(10) Pub. No.: US 2007/0122541 A1
(43) Pub. Date: May 31, 2007

(54) Method for preparation of bioactive ceramic-coated composite

(76) Inventors: Eungje Lee, Seoul (KR); Young Min Kong, Daejeon-city (KR); Jongsik Choi, Daejeon-city (KR)

Correspondence Address: MCKENNA LONG & ALDRIDGE LLP, 1900 K STREET, NW, WASHINGTON, DC, 20006, US

(21) Appl. No.: 10/563,254

(22) PCT Filed: Oct. 5, 2005
(86) PCT No.: PCT/KR05/03277

§ 371 Date: Jan. 4, 2006

(30) Foreign Application Priority Data
Oct. 5, 2004 (KR) 10-2004-0079203

(51) Int. Cl. A61L 27/32; C23C 14/32; C23C 16/00; B05D 3/12; B05D 1/18

(52) U.S. Cl. 427/2.27; 427/430.1; 427/248.1; 427/355; 204/192.1

(57) ABSTRACT

Provided is a method of preparing a bioactive ceramic-coated composite. The method includes coating calcium phosphate-based ceramic on a ceramic substrate; and thermally treating the coated calcium phosphate-based ceramic layer in a water vapor atmosphere. The bioactive ceramic-coated composite prepared using the method in which the decomposition of hydroxyapatite is suppressed can be used for artificial bioactive tissues which are harmless to the human body and satisfy chemical and mechanical requirements.

### BACKGROUND OF THE INVENTION

1. Field of the Invention

The present invention relates to a method of preparing a bioactive ceramic-coated composite, and more particularly, to a method of preparing a bioactive ceramic-coated composite, which is harmless to the human body and satisfies mechanical and chemical requirements.

2. Description of the Related Art

Up to now, ceaseless studies on artificial tissues, which are similar to hard tissues such as bones, teeth, and joints of the human body, do not cause biological side effects, and can be naturally used without causing any chemical and mechanical problems. The history of artificial tissues begins with metals having excellent mechanical properties, such as stainless steel or chrome-cobalt steel. However, metals with excellent mechanical properties gradually corrode in the highly corrosive body fluid and produce metal ions, which diffuse into all organs of the human body, thus causing inflammations or cancers. Also, because a metal, such as stainless steel and chrome-cobalt steel, has no affinity to living organs, xenobiotics, such as a fibrous film, are formed on the surface of the metal, and the metal cannot bind to adjacent bones and rather destroys the bones. Therefore, a patient must undergo additional surgery after a predetermined duration of time has passed.

To solve the problems arising with such metals, research into ceramics has been performed. Much attention has been paid to alumina (Al₂O₃) and zirconia (ZrO₂), which are ceramics having good mechanical characteristics. Although these ceramics are incorrodible unlike metals, they still do not directly bind to bones and form a fibrous film on the interface between the ceramic and the bone.

Meanwhile, bio-ceramics that directly combine with bones were developed. Examples of such bio-ceramics include CaO—SiO₂-based bioactive glass, crystalline glass, a calcium phosphate compound containing apatite, which is a bone component, etc. These bio-ceramics directly combine with bones and cause neither inflammation nor xenobiotic reaction at interfaces. However, since the mechanical strength and the fracture toughness of the bio-ceramics are poor, they cannot be used as artificial bones for parts which are resistant to a high stress, like teeth, or parts requiring high mechanical strength and fracture toughness, such as a hip joint. For this reason, apatite has limited applications in a few parts, like auditory ossicles that do not require high mechanical strength.

Furthermore, a method of using apatite-wollastonite (CaO.SiO₂) glass-ceramics (A/W glass-ceramics) instead of metals is proposed. The mechanical strength of the A/W glass-ceramics is slightly higher than sintered apatite but is still insufficient for wild applications.

To overcome the above-described drawbacks of metals, there have been attempts in recent years to coat a bioactive ceramic layer on a metal. This method utilizes the mechanical strength and fracture toughness of metals and the biocompatibility and bioactivity of apatite. However, because of poor interfacial adhesion between the ceramic-coated layer and a metal, the ceramic-coated layer endures repeatedly applied loads and is eventually separated. The separation of the ceramic-coated layer induces inflammations and necrosis of tissues and thus is suppressed.

In order to prevent the separation of an apatite-coated layer and a metallic material due to differences in mechanical and thermal properties, methods of coating apatite on a ceramic substrate have been proposed. As an example, according to U.S. Pat. No. 5,077,079, only calcium metaphosphate (CaP₂O₆) or a mixture with calcium pyrophosphate (Ca₂P₂O₇) is coated on a ceramic substrate and thermally treated until it is fixed to the ceramic substrate as an intermediate layer. Thereafter, a slurry mixture of CaP₂O₆ and tricalcium phosphate (TCP) (Ca₃(PO₄)₂) is coated on the intermediate layer and thermally treated to densify the coated layer. Also, there is another method taught in U.S. Pat. No. 5,472,734 in which calcium salt is coated on an alumina ceramic substrate, and the resultant is immersed in a phosphoric acid solution containing phosphate to modify it into apatite. Further, Korean Patent Publication No. 2000-18897 discloses a method of coating a thin hydroxyapatite layer, in which hydroxyapatite to which a calcium compound is added and a target to be coated with the hydroxyapatite are loaded in a chamber with an electron gun and an ion gun, the chamber is evacuated, and ions are jet onto the material layer using the ion gun to vaporize the hydroxyapatite and form the hydroxyapatite layer on the target.

In addition, Korean Patent Publication No. 10-424,910 discloses a method of coating apatite on a ceramic material, such as zirconia or alumina. This method of coating a bioactive ceramic includes dispersing bioactive ceramic powder, which is used for an artificial biomaterial, in a solvent together with a binder to obtain a slurry and coating the slurry on a ceramic oxide substrate. Artificial teeth or bone marrow transplantation using the coating method is also disclosed in the patent.

Moreover, Korean Patent Laid-open Publication No. 10-2004-1325 discloses a method of suppressing a reaction between hydroxyapatite and secondary phase by substituting hydroxy ions of hydroxyapatite with fluoride ions. More specifically, apatite does not dehydrate and decompose even after being sintered and does not form undesired materials, such as TCP, tetracalcium phosphate (TTCP), calcium oxide, etc., thereby preventing deterioration of bioactive and mechanical properties of an apatite composite.

Furthermore, Japanese Patent Laid-Open Publication No. 6-60069 discloses an apatite coating composite material and a method of preparing the same. In particular, a slurry mixture of calcium metaphosphate (CaP₂O₆) and TTCP is coated, exposed to water vapor for a sufficient duration of time, and thermally treated at a high temperature. As a result, β-TCP is generated along with hydroxyapatite, thereby resulting in a denser coated layer.

However, the above-described conventional methods involve complicated processes and preclude the formation of a 100% apatite-coated layer.

### SUMMARY OF THE INVENTION

The present invention provides a method of preparing a bioactive ceramic-coated composite by coating a calcium phosphate-based ceramic layer on a ceramic substrate, thus preventing the deterioration of mechanical and chemical properties of the bioactive ceramic-coated composite caused by decomposition of hydroxyapatite.

According to an aspect of the present invention, there is provided a method of preparing a bioactive ceramic-coated composite, the method including coating calcium phosphate-based ceramic on a ceramic substrate and thermally treating the coated calcium phosphate-based ceramic layer while supplying water vapor.

### BRIEF DESCRIPTION OF THE DRAWINGS

The above and other features and advantages of the present invention will become more apparent by describing in detail exemplary embodiments thereof with reference to the attached drawings in which:

FIG. 1 is an XRD spectrum of a bioactive ceramic-coated layer prepared in Example 1 according to the present invention;

FIG. 2 is an XRD spectrum of a bioactive ceramic-coated layer prepared according to Comparative Example 1; and

FIG. 3 is a graph of cellular reactivity of the bioactive ceramic-coated layers according to Example 1 and Comparative Example 2.

### DETAILED DESCRIPTION OF THE INVENTION

The present invention will now be described more fully hereinafter with reference to the accompanying drawings.

The present invention provides a method of preparing a bioactive ceramic-coated composite, which includes coating a calcium phosphate-based ceramic layer on a ceramic substrate and thermally treating the coating layer while supplying water vapor.

When zirconia is used as a ceramic substrate, and hydroxyapatite is used as ceramic powder, the hydroxyapatite decomposes during the thermal treatment as shown in Reaction schemes (1) and (2), thus generating tricalcium phosphate (TCP) secondary phase.
Ca₁₀(PO₄)₆(OH)₂+ZrO₂⇄3Ca₃(PO₄)₂+H₂O(g)↑t+CaO+ZrO₂ (1)
CaO+ZrO₂→CaZrO₃ (2)
In a conventional method, while the bioactive ceramic-coated layer is thermally treated, it reacts with the component of the ceramic substrate, i.e., zirconia, and decomposes, thus generating TCP as shown in Reaction scheme (1).

This reaction increases the solubility of the coated layer and lowers the bio-activation thereof. As a result, a desired ceramic composite cannot be obtained.

In the present invention, by supplying water vapor, which is one of products from Reaction scheme (1), into a reactor in which the reaction in Reaction scheme (1) occurs, the reaction equilibrium is shifted toward reactants, thereby suppressing decomposition of hydroxyapatite into TCP.

In the present invention, the calcium phosphate-based ceramic layer may be formed of hydroxyapatite, fluoroapatite, tricalcium phosphate (TCP), tetracalcium phosphate (TTCP), calcium phosphate, or tetracalcium hexaphosphate. Among these examples, hydroxyapatite, fluoroapatite, and TTCP are preferred in view of bioactivity, and hydroxyapatite is most preferred.

When fluoroapatite is thermally treated without water vapor, TCP is generated in the same manner as hydroxyapatite. However, when fluoroapatite is thermally treated in a water vapor atmosphere, it changes into fluoro-hydroxyapatite as shown in Reaction scheme (3) below.
Ca₁₀(PO₄)₆F₂→Ca₁₀(PO₄)₆(OH, F)₂ (3),
where Ca₁₀(PO₄)₆(OH, F)₂ indicates that fluorine is partially substituted by hydroxyl group.

In addition, when TTCP(Ca₄(PO₄)₂O) is thermally treated in a water vapor atmosphere, hydroxyapatite is generated as shown in Reaction scheme (4) below.
3Ca₄(PO₄)₂O+H₂O→Ca₁₀(PO₄)₆(OH)₂+2CaO (4)

After fluoro-hydroxyapatite or hydroxyapatite is generated as in Reaction schemes (3) and (4), the decomposition of fluoro-hydroxyapatite or hydroxyapatite into TCP is prevented due to the ambient water vapor for the same reason as described with reference to Reaction scheme (1).

The ceramic substrate may be an alumina (Al₂O₃) substrate, a zirconia substrate, or a titania substrate. The alumina substrate or the zirconium substrate is preferred because they have a ceramic structure with good mechanic properties.

A method of coating the calcium phosphate-based ceramic layer on the ceramic substrate may be performed using a variety of methods that are known to those skilled in the art. Examples of the methods include a dipping method, a tape casting method, a doctor blade method, etc., in which a slurry of calcium phosphate-based ceramic is prepared and coated on the surface of a ceramic substrate, and a biomimetic coating process, a physical vapor deposition (PVD) process, a chemical vapor deposition (CVD) process, a plasma spray process, etc., in which a slurry is not used.

According to a slurry coating process, initially, a slurry in which calcium phosphate-based ceramic to be coated on a ceramic substrate is dispersed is prepared using a method known to those skilled in the art. For example, the slurry is prepared by adding calcium phosphate-based ceramic powder in a solvent, such as ethanol or water, and mixing and milling the solution. Here, a binder such as polyvinyl alcohol (PVA) or polyvinyl butyral (PVB) is added to adjust the viscosity of the slurry, and a dispersant is added to prevent the agglomeration of the slurry and improve the dispersion stability of the slurry.

The slurry prepared as described above is coated on the ceramic substrate using a suitable method selected from among the above-described methods.

The thickness of the slurry coating layer may be adjusted to be about 0.1 μm to 1 mm. When the thickness of the coating layer is less than 0.1 μm, the binding force of the coating layer to body tissue is weakened. When the thickness of the coating layer is greater than 1 mm, the stress concentrates on the coating layer having a small mechanical strength, and thus the coating layer cracks or is broken. The thickness of the coating layer may be controlled by varying the amount of ceramic powder in the coating solution or by repeating a coating process.

After the slurry is coated on the surface of the ceramic substrate as described above, the slurry is dried at a temperature of about 15 to 95° C. for 5 to 12 hours. Preferably, the slurry coating layer is firstly dried at room temperature for a predetermined duration, and then the drying temperature is slowly raised. If the slurry coating layer is dried at a high temperature from the beginning, it cracks due to the high drying rate. Also, if the slurry coating layer is dried at a temperature of 95° C. or higher, the polymeric components in the slurry may decompose.

The dried coating layer and ceramic substrate are thermally treated at a temperature of 500-800° C. to burn out the polymer used as a binder to sinter the coating layer. The temperature of the reactor may be gradually raised at a rate of 0.01 to 5° C./min. When the temperature raising rate is too high, the polymer abruptly burns and the coating layer loses the shape.

When the polymer burns out, only ceramic remains in the coating layer. To further densify the ceramic, the ceramic material is sintered at a temperature of 1000° C. or higher to obtain a final ceramic-coated composite.

Alternatively, a biomimetic coating process can be used in the present invention. This method fundamentally utilizes a heterogeneous nucleation process.

Initially, a calcium source and a phosphate source, which are raw materials for forming hydroxyapatite, are melted in distilled water in an appropriate ratio. Here, the molar ratio of calcium to phosphate is set to 1.67, which corresponds to a molar ratio of hydroxyapatite, such that the concentration of the resulting solution is supersaturated. A ceramic substrate whose surface is activated by being processed using an acid or a base is dipped in the solution having the above composition for several hours to several days. Thus, hydroxyapatite crystals are grown throughout the ceramic substrate so that a hydroxyapatite coating layer is completed.

Alternatively, a PVD process can be used in the present invention. To be specific, a hydroxyapatite target is loaded into a vacuum chamber and deposited on a substrate using electronic beams, ion beams, or plasma. Examples of a method used to deposit hydroxyapatite on the substrate include a sputtering process, an evaporation process, a laser ablation process, etc.

Alternatively, a plasma spray process or a thermal spray process can be used in the present invention. These processes are most commonly used for commercial purposes to form a hydroxyapatite coating layer. In particular, hydroxyapatite powder is melted using plasma (or heat) and sprayed onto a ceramic substrate to coat a hydroxyapatite layer thereon.

TCP(Ca₃(PO₄)₂), which is a secondary phase main component, decreases the bio-activity of the coating layer and increases the solubility of the coating layer, thus degrading chemical and mechanical stabilities of the coating layer. For this reason, the generation of TCP has to be suppressed. Accordingly, when injecting water vapor, which is one of products in Reaction scheme (1), during a thermal treatment process, the equilibrium of Reaction scheme (1), which is a reversible reaction, shifts closer to reactants than when no water vapor is injected, so that the generation of TCP is naturally suppressed.

The thermal treatment may be performed at a temperature of about 800 to 1800° C. When the thermal treatment is performed at a temperature lower than 800° C., the coating layer is not sintered so that reliable adhesion of the coating layer to the ceramic substrate cannot be obtained. Meanwhile, when the thermal treatment is performed at a temperature higher than 1800° C., the operation costs are too high, and hydroxyapatite is highly likely to decompose into TCP due to the high temperature even in a water vapor atmosphere.

The partial pressure of the injected water vapor may be in a range of 10⁻⁴ to 1 atmospheric pressure at room temperature. When the partial pressure of the water vapor is lower than 10⁻⁴ atmospheric pressure, hydroxyapatite decomposes into TCP, which does not comply with the purpose of injecting water vapor. Meanwhile, when the partial pressure of the water vapor is higher than 1 atmospheric pressure, the pressure rises too high at a high temperature, and the manufacturing costs of the reactor increase. A supply system for supplying water vapor may be constructed such that oxygen, nitrogen, or argon passes through water above the coating layer or such that water vapor generated by boiling water can be supplied to the coating layer.

Hereinafter, the present invention will be described in greater detail with reference to the following examples. The following examples are for illustrative purposes only and are not intended to limit the scope of the present invention.

### EXAMPLE 1

14 g of hydroxyapatite powder was added to 100 ml of ethanol and dispersed. 1 g of TEP was added as a dispersant to prevent the agglomeration of the powder and improve the dispersion stability, and 1 g of PVB was added as a binder to adjust the viscosity of a slurry. To uniformly disperse hydroxyapatite powder in the mixture and reduce the particle size of the powder, the mixture was milled using zirconia balls for 24 hours to obtain the slurry.

A sintered zirconia substrate was coated by being dipped in the prepared slurry for about 3 seconds, slowly taken out of the slurry. The thickness of a coated layer on the zirconia substrate, which varies according to the viscosity and the particle size distribution of the slurry, was controlled to be 0.5 to 10 μm after a single coating process. The thickness of the coated layer could be controlled through repeated coating processes. The resulting coated structure was dried in a thermostatic drier at 80° C. for 12 hours.

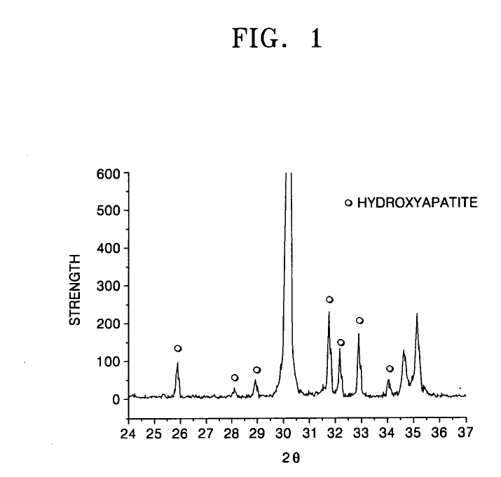

The dried coated structure was loaded into an electric furnace. Thereafter, in order to create a water vapor atmosphere in the electric furnace, oxygen discharged at a gauge pressure of 60 mmHg was incorporated into distilled water and supplied into the electric furnace. That is, the discharged oxygen gas incorporated into the distilled water served as a carrier gas for supplying water molecules into the electric furnace. While maintaining the water vapor atmosphere as described above, the temperature of the electric furnace was raised at a rate of 2° C./min to 800° C. and then maintained at the same temperature for 5 hours until polymer burnt out. In order to sinter the hydroxyapatite coated layer in which the polymer did not remain as a result of the burning, the temperature of the electric furnace was raised at a rate of 2° C./min up to 1200° C. and then maintained at the same temperature for 1 hour, thereby completing a sintering process. Thereafter, the hydroxyapatite coated layer was cooled at a constant cooling rate of 2° C./min to minimize generation of cracks caused by a difference in thermal expansion coefficient coefficient between the coated layer and the substrate. As a result, the zirconia substrate with the hydroxyapatite layer coated thereon was obtained. An XRD spectrum of the resultant structure is illustrated in FIG. 1.

### COMPARATIVE EXAMPLE 1

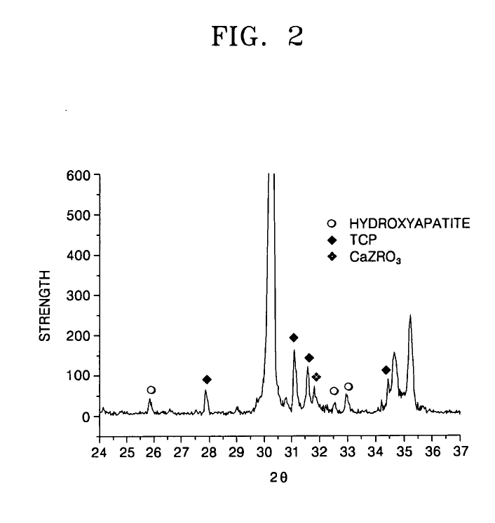

A zirconia substrate with hydroxyapatite coated layer was obtained under the same experimental conditions as in Example 1, except that no water vapor was injected. An XRD spectrum of the resultant structure is illustrated in FIG. 2.

As can be seen from FIGS. 1 and 2, when a hydroxyapatite layer was thermally treated in a water vapor atmosphere, secondary phases such as TCP and CaZrO₃ were not generated.

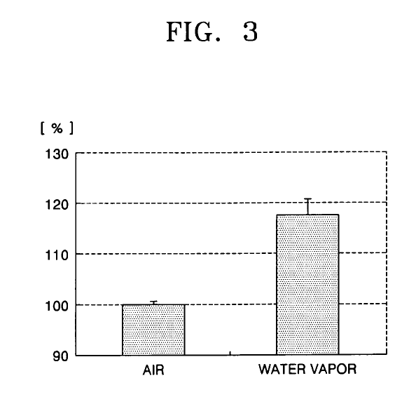

Also, a cellular experiment was carried out using the hydroxyapatite ceramic-coated composites prepared in Example 1 and Comparative Example 1. Specifically, osteoblast cells, which form bones, were cultivated on each of the ceramic-coated composites for 3 days, and the amount of proliferated cells was measured. As a result, as shown in FIG. 3, when the number of cells cultivated on the ceramic-coated composite prepared in an air atmosphere is defined as 100, the number of cells cultivated on the ceramic-coated composite prepared in the water vapor atmosphere is about 117, which is a 17% increase over the number of cells cultivated in the air atmosphere.

As described above, a bioactive ceramic-coated composite according to the present invention has excellent chemical and mechanical stabilities because the decomposition of hydroxyapatite during a thermal treatment process is suppressed. The bioactive ceramic-coated composite according to the present invention, which is mechanically and chemically stable, can be used for artificial bioactive tissues which are harmless to the human body and satisfy chemical and mechanical requirements.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method of preparing a bioactive ceramic-coated composite, the method comprising:
coating calcium phosphate-based ceramic on a ceramic substrate; and
thermally treating the coated calcium phosphate-based ceramic layer while supplying water vapor.

2. The method according to claim 1, wherein the ceramic substrate is formed of at least one material selected from the group consisting of zirconia, alumina, and titania.

3. The method according to claim 1, wherein the coated layer has a thickness of about 0.1 μm to 1 mm.

4. The method according to claim 1, wherein the calcium phosphate-based ceramic layer is formed of at least one material selected from the group consisting of hydroxyapatite, fluoroapatite, tricalcium phosphate, tetracalcium phosphate, calcium phosphate, and tetracalcium hexaphosphate.

5. The method according to claim 1, wherein the coating of the calcium phosphate-based ceramic layer is performed using at least one selected from the group consisting of a dipping process, a doctor blade process, a physical vapor deposition (PVD) process, a chemical vapor deposition (CVD) process, and a biomimetic coating process.

6. The method according to claim 1, wherein the thermally treating of the coated layer is performed at a temperature of about 800 to 1800° C.

7. The method according to claim 1, wherein the supplying of the water vapor is performed under a partial pressure of 10⁻⁴ to 1 atmospheric pressure.

8. The method according to claim 1, wherein the supplying of the water vapor comprises incorporating at least one gas selected from the group consisting of oxygen (O₂), nitrogen (N₂), and argon (Ar) into water and flowing the gas above the coated layer.

9. The method according to claim 1, wherein the supplying of the water vapor comprises supplying the water vapor generated by boiling water to the coated layer.
